(19) United States
(12) Patent Application Publication
(10) Pub. No.: US 2015/0202306 A1
(43) Pub. Date: Jul. 23, 2015

(54) OPHTHALMIC COMPOSITIONS WITH WAX ESTERS

(71) Applicant: Bausch & Lomb Incorporated, Rochester, NY (US)

(72) Inventor: Martin J. Coffey, Buffalo Grove, IL (US)

(21) Appl. No.: 14/416,229

(22) PCT Filed: Nov. 13, 2012
(86) PCT No.: PCT/US2012/064791

§ 371 Date: Jan. 21, 2015

Related U.S. Application Data
(63) Continuation of application No. 13/600,723, filed on Aug. 31, 2012, now abandoned.

(51) Int. Cl. A61K 47/44; A61K 47/32; A61K 47/24; A61K 9/00

(52) U.S. Cl. CPC A61K 47/44; A61K 9/0048; A61K 47/32; A61K 47/24

(57) ABSTRACT

A suspension comprising a mixture of wax esters suspended in a formulation vehicle. The formulation vehicle comprises a lightly cross-linked carboxy-containing polymer and a concentration of ionic salt components to provide the suspension with a calculated ionic strength of less than 0.1. The suspension has the following rheological properties, (T>G″ and a suspension yield value of greater than 1 Pa. Also, upon addition of 30 mL of the suspension to a volume of 6 mL to 12 mL of simulated tear fluid, the resulting tear mixture transitions to a liquid form wherein, G″>G′ and the tear mixture has a yield value of less than 0.1 Pa.

### BACKGROUND

The present invention relates to ophthalmic compositions that include a mixture of wax esters suspended in an aqueous gel formulation vehicle, and a medical use of the compositions to alleviate symptoms associated with dry eye or other ocular disorders.

Ophthalmic compositions are used to provide relief of a variety of ocular conditions and ocular disease states. In most instances, ophthalmic compositions are administered or instilled to the eye via eye drops from a multi-dose container in the form of solutions, ointments or gels. If the ophthalmic active component is soluble, or even slightly soluble, in water, a formulator may proceed with a solution eye drop product. However, if the solution product has to low of a viscosity; e.g., less than about 30 cp (or mPa s), upon instillation the ophthalmic active can be rapidly discharged from the precorneal area of the eye because of lacrimal secretion and nasolacrimal drainage. As a result, it has been estimated that approximately 80-99% of the ophthalmic active component is simply washed or flushed from the eye before the active actually contacts the desired ocular tissue to achieve its desired clinical effect. The poor residence time of the active in the eye thus requires frequent instillation or use of a more concentrated active product to achieve the desired clinical effect. To lengthen the residence time of ophthalmic active, and thus, to enhance the bioavailability of the ophthalmic active per instillation, non-solution based ophthalmic vehicles have been developed. Examples of such ophthalmic vehicles include ointments or stabilized emulsions. However, these ophthalmic vehicles can have their drawbacks as well. For example, the use of ointments often causes blurred vision just after instillation. In some instance, the patient can sense a “goopy feeling” in their eyes, which, of course, is also undesirable.

Some ophthalmic formulators have resorted to the so-called in situ gel-forming systems. These ophthalmic vehicles can extend precorneal residence time and improve ocular bioavailability of the ophthalmic active. Typically, in situ gel-forming systems are usually aqueous solutions and contain one or more polymers. The ophthalmic products tend to exist as a low-viscosity liquid during storage in the dispenser container and form a gel upon contact with tear fluid. The liquid-to-gel transition can be triggered by a change in temperature, pH, ionic strength, or the presence of tear proteins depending on the particular polymer system employed.

For example, A. Rozier et al., Int. J. Pharm. (1989), 57: 163-168, discloses a composition comprising an ion-activated gelling gellan gum (a polysaccharide) with the tradename of Gelrite® and an ion content below the gelation concentration. Rozier et al.'s gellan gum composition rapidly gels when mixed with simulated tear fluid having a combined concentration of mono- and divalent cations (sodium and calcium) of about 0.14 M. U.S. Pat. No. 5,192,535 discloses an aqueous ophthalmic composition comprising a crosslinked carboxy-containing polymer. The composition has viscosity in the range of 1,000-30,000 cp and pH of 3-6.5, which rapidly gels (to viscosity of 75,000-500,000 cp) upon contact with the higher pH of tear fluid. Joshi et al.'s U.S. Pat. No. 5,252,318 discloses reversibly gelling aqueous compositions which contain at least one pH-sensitive reversibly gelling polymer (such as carboxy vinyl linear or branched or cross-linked polymers of the monomers) and at least one temperature-sensitive reversibly gelling polymer (such as alkylcellulose, hydroxyalkyl cellulose, block copolymers of polyoxyethylene and polyoxypropylene, and tetrafunctional block polymers of polyoxyethylene and polyoxypropylene and ethylenediamine). It is contemplated that a high amount of salt (up to 0.2-0.9%) is used to have a low viscosity in the ungelled state. The compositions are formulated to have a pH of 2.5-6.5; preferably, 4-5.5. The viscosity of the compositions increases by several orders of magnitude (up to 1,000,000 cp) in response to substantially simultaneous changes in both temperature and pH.

U.S. Pat. No. 6,511,660 discloses a composition comprising Carbopol and Pluronic® (a polyoxyethylene-polyoxypropylene copolymer) formulated at pH of 4. The composition turns into a stiff gel when in contact with physiological condition (37° C. and pH of 7.4). Kumar et al., J. Ocular Pharmacol., Vol. 10, 47-56 (1994), discloses an ocular drug delivery system based on a combination of Carbopol and methylcellulose, prepared at pH of 4. This system turns into a stiff gel when the pH is increased to 7.4. Kumar et al., J. Pharm. Sci. Vol. 84, 344-348 (1995), discloses yet another ocular drug delivery system containing Carbopol and hydroxyproplymethylcellulose, also prepared at pH of 4. This system turns into a stiff gel when the pH is increased to 7.4 and the temperature to 37° C. In both systems, a viscosity-enhancing polymer (methylcellulose or hydroxypropylmethylcellulose) is added in order to not have excessive amount of Carbopol® concentration without compromising the in situ gelling properties as well as overall rheological behaviors. Finkenaur et al.'s U.S. Pat. No. 5,427,778 discloses gel formulations that contain a polypeptide growth factor and a water soluble, pharmaceutically or ophthalmically compatible polymeric material for providing viscosity within various ranges determined by the application of the gel.

The above prior-art ophthalmic compositions all have a common characteristic of having a low viscosity in the dispenser container and becoming a stiff gel upon being instilled in the eye due to an increase in at least one of pH, temperature, and ionic strength. Although a stiff gel can have an extended residence in the eye and assist in promoting a higher drug bioavailability, and perhaps enhance clinical outcome per instillation, such gels, like the ointments, can interfere adversely with vision and result in patient dissatisfaction. In addition, these prior-art compositions must often be formulated at significantly acidic pH, which is not comfortable upon installation in the eye of the patient.

The surface of the eye requires constant lubrication for proper function. This includes quality of vision as well as comfort. The eye becomes irritated and vision blurs when inadequately lubricated. This condition is frequently referred to as dry eye. Dry eye is a common ocular condition and many over-the-counter and prescription therapies are available to mitigate this difficult to treat and uncomfortable condition. Many patients, however, are unable to find relief with present therapies.

It is well recognized that the meibomian gland secretions of the eyelid provide the lipid layer of the tear film. The major component of the meibomian gland lipid secretions are wax esters (Driver and Lemp, Meibomian Gland Dysfunction, Surv Ophthalmol 40:343-367, 1996). Various wax esters have been identified in the secretions of Meibomian glands (meibum) (Butovich I A et al., Lipids 2007, 42, 765). The three most abundant species were C18:1 fatty acid esters of C24:0, C25:0, and C26:0 fatty alcohol. Typically, a major was species based on C18:1 fatty acid and a saturated fatty alcohol was accompanied by a few related compound based on a C18:2, C18:3, and C18:4 fatty acid (Butovich I A et al., J Lipid Res 2009, 50, 2471). Meibum is an intrinsic part of the human tear film, the main role of which is to protect the ocular surface from dehydration. Among other proposed functions of the tear film are antimicrobial, lubricant, and nutritional ones.

### SUMMARY OF THE INVENTION

An ophthalmic composition comprising a mixture of wax esters suspended in a formulation vehicle, the vehicle comprising a lightly cross-linked carboxy-containing polymer and a concentration of ionic salt components to provide the suspension with a calculated ionic strength of less than 0.1. The suspension has the following rheological properties, G′>G″ and a suspension yield value of greater than 1 Pa. Also, upon addition of 30 mL of the suspension to a volume of 6 mL to 12 mL of simulated tear fluid, the resulting tear mixture transitions to a liquid form wherein, G″>G′ and the tear mixture has a yield value of less than 0.1 Pa.

A suspension comprising a mixture of wax esters suspended in a formulation vehicle, the vehicle comprising a lightly crosslinked carboxy-containing polymer and a concentration of ionic salt components to provide the suspension with a calculated ionic strength of from 0.03 to 0.08. The suspension has the following rheological properties, G′>G″ and a suspension yield value of from 2 Pa to 8 Pa. Also, upon addition of 30 mL of the suspension to a volume of 10 mL of simulated tear fluid to provide a tear mixture of the suspension in a simulated ocular condition, the tear mixture has a tear mixture yield value from 0 Pa to 0.1 Pa and a tear thin value of from 5 to 30.

A method for suspending a mixture of wax esters in an aqueous-based, ophthalmic suspension. The method comprises combining the ophthalmic active with a formulation vehicle, the vehicle comprising a lightly crosslinked carboxy-containing polymer and a concentration of ionic salt components to provide the suspension with a calculated ionic strength of from 0.03 to 0.08. The suspension has the following rheological properties, G′>G″, a suspension yield value of from 2 Pa to 8 Pa, and upon addition of 30 mL of the suspension to a volume of 10 mL of simulated tear fluid to provide a tear mixture of the suspension in a simulated ocular condition, the tear mixture has a tear mixture yield value of less than 0.1 Pa and a tear thin value of from 5 to 30.

A unit dosage package for administration of an ophthalmic composition in the form of an eye drop, the ophthalmic composition comprising a mixture of wax esters suspended in a formulation vehicle, the formulation vehicle comprising a lightly crosslinked carboxy-containing polymer and a concentration of ionic salt components to provide the suspension with a calculated ionic strength of from 0.03 to 0.08. The ophthalmic formulation has the following rheological properties, G′>G″, and a suspension yield value of from 2 Pa to 8 Pa, and upon addition of 30 mL of the suspension to a volume of 10 mL of simulated tear fluid to provide a tear mixture of the suspension in a simulated ocular condition, the tear mixture has a tear mixture yield value from 0 Pa to 0.1 Pa and a tear thin value of from 5 to 30.

### DETAILED DESCRIPTION OF THE INVENTION

Due to the unique physiological and biomechanical conditions of the eye formulating ophthalmic compositions to optimize clinical efficacy and patient compliance, yet minimize or avoid patient dissatisfaction following instillation in the form of drops remains a great challenge. The challenge is heightened considerably with ophthalmic compositions that include a mixture of wax esters. Due to the limited, or near non-existent, solubility of wax esters in water, the wax esters must be suspended in a vehicle, typically as an oil-in-water emulsion or ointment. In such instances, however, it is very difficult to formulate wax esters to maintain a substantially uniform suspension or distribution in the formulation vehicle in order to have a consistent unit (instillation) dosage. In nearly all instances, a patient will have to vigorously shake the product (much like an inhaler used by asthma patients) to best ensure a consistent and accurate dosage. For this reason, it is especially difficult to suspend a mixture of wax esters in an aqueous vehicle formulation for drop instillation that does not require a pre-shaking of the product. The formulation vehicle described herein addresses these shortcomings with present ophthalmic suspension formulations.

As used herein, the term “mixture of wax esters” is a mixture of compounds that comprise an ester linkage positioned between two long aliphatic chains, saturated or partially unsaturated, each chain having at least twelve (12) carbons, e.g., C12 to C34.

In one embodiment, the mixture of wax esters can include one or more compounds of wax esters with a glyceride core. In another embodiment, the mixture of wax esters will have less than 20 percent by weight of one or more compounds of wax esters with a glyceride core based upon the total weight of wax esters in the composition. In many such instances, the mixture of wax esters will have less than 6 percent by weight of one or more compounds of wax esters with a glyceride core based upon the total weight of wax esters in the composition.

In one preferred embodiment, the mixture of wax esters is derived or extracted from a natural wax, the compounds of which result from a condensation of a long-chain fatty alcohol with a long-chain fatty acid. The natural sources of wax esters include, but are not limited to, beeswax, jojoba, carnauba and lanolin. One advantage of the naturally derived wax esters is that the mixture of wax esters would have less than 10% of wax esters with a glyceride core (see below).

As used herein, use of the term the “solubility in water” of an organic compound, including a mixture of was esters, in water means the compound has a solubility in water as measured at 25° C. and pH of 7 of less than 0.1 times the concentration of the compound in mg/mL in the ophthalmic composition. For example, if the compound is present in an ophthalmic composition at a concentration of 0.1 mg/mL, the compound will have a solubility in water at 25° C. and a pH of 7 of less than 0.1(0.1 mg/mL), which is less than 0.01 mg/mL. Likewise, for a compound that is present in an ophthalmic composition at a concentration of 10 mg/mL, the compound will have a solubility in water at 25° C. and a pH of 7 of less than 0.1(10 mg/mL), which is less than 1.0 mg/mL. Accordingly, the term “solubility in water” refers to the water solubility of a compound in an ophthalmic composition as well as the compounds concentration in the composition in mg/mL. For example, a mixture of wax esters present at a relatively high concentration in an ophthalmic composition can have a somewhat greater water solubility than a different mixture of wax esters with a lower water solubility present in another composition at a lower concentration, but because of the higher concentration of the mixture of wax esters in the former composition a significant portion of the mixture remains suspended in the composition.

The described ophthalmic formulation vehicle provides a storage-stable, suspension of a mixture of wax esters in the form of a gel. However once instilled into the eye via one or more eye drops, the gel transitions to a liquid form, i.e., it loses its gel character. This transition from gel to liquid is important for patient compliance because of the dissatisfaction patients express after having instilled ophthalmic gels or ointments. These prior art vehicle formulations remain for a period of time in the eye as gels, particularly over the initial 1 to 3 minutes following instillation, and cause visual impairment. The gels or ointments can also cause ocular discomfort, which can lead to patients skipping one or more of a scheduled dosing regimen. The term “storage-stable” means that a stirred or shaken composition of a mixture of wax esters in the described formulation vehicle will provide a suspension of the wax esters in the formulation vehicle, and the wax esters will remain effectively suspended in the formulation vehicle for at least two weeks, and in many cases, for up to four weeks or even eight weeks, without having to stir or shake the drug product in its packaged container. The term “effectively suspended” means an ophthalmic suspension formulation that delivers 90% to 110% of a predetermined dosage of a mixture of wax esters per eye drop without a patient having to purposefully shake the drug product container more than once every two weeks. Why is the storage-stability of an ophthalmic suspension so important? Because with non-storage-stable formulations many patients forget to shake the product before instillation. As a result, the patient is not instilling a consistent and proper dosage. This can be a problem because after the first twenty drops or so each subsequent eye drop can contain greater concentrations of whatever one is attempting to deliver to the eye, which may not be a good thing.

### Jojoba Wax Esters

Jojoba wax is extracted from seeds and leaves of the jojoba tree (Simmondsia chinensis) cultivated in the desert conditions of the American Southwest and other locations. Jojoba wax has a melting point of about 6° C., and its chemical structure typically does not vary with plant type, growing location, soil type, rainfall or altitude. The extract produced from jojoba includes a mixture of wax esters, which is actually in the form of a liquid wax, that protects the shrub from its severe arid natural habitat. Jojoba wax or the mixture of wax esters thus keep the shrub well lubricated and moisturized.

The natural jojoba is 97% wax esters with few impurities. The components of the jojoba wax esters include long chain alcohols esterified with long chain fatty acids with a total of 38 to 44 carbon atoms with one double bond in each alkyl moiety. Jojoba wax esters comprise primarily 18:1 (6%), 20:1 (35%) and 22:1 (7%) fatty acids linked to 20:1 (22%), 22:1 (2)%) and 24:1 (4%) fatty alcohols. Exemplary long chain fatty acids include gadoleic, palmitic, palmitoleic, stearic, oleic, linoleic, arachidic, linolenic, eicosenoic, behenic, erucic, lignoceric, lactic, decate, acetic and myristic fatty acids. The fatty acids typically have carbon chains of C12 to C30, with or without various degrees of saturation or unsaturation. The alcohol components of the wax ester contain carbon chains between C16 and C32 with or without various degrees of saturation or unsaturation. The alcohol component may be eicos-11-enol, docos-13-enol, tetracos-15-enol, myristyl alcohol, octyldodecyl stearoyl alcohol or cetyl alcohol.

Other jojoba derived mixture of wax esters include jojoba esters, which are the result of an inter-esterification of various ratios of jojoba liquid wax and hydrogenated jojoba solid wax. The physical consistency ranges from liquid to semisolid paste or creams. Jojoba solid wax is derived from the hydrogenation and complete reduction of the unsaturated wax esters. It is a hard crystalline wax comparable to beeswax with a melting point of 69° C. Jojoba alcohols are generated from a sodium reduction of jojoba liquid wax and hydrogenated jojoba solid wax with subsequent additional refinement. Jojobutter-51 is an isomorphous mixture of jojoba liquid wax, partially isomerized jojoba liquid wax and hydrogenated jojoba solid wax (J Amer College Toxicology, 11 (1), 1992).

### Other Natural Wax Esters

Beeswax is an abdominal secretion of bees (Apis mellifera), and is what bees use to form and seal the hive cells. Beeswax is easily saponifiable and emulsifiable because of its content in free fatty acids, diols and hydroxyacids. Beeswax is primarily a mixture of palmitate, palmitoleate, hydroxypalmitate and oleate esters of long-chain alcohols (C30-32) (about 70 to 80% of the total weight). The ratio of triacontanylpalmitate (or melissylpalmitate, C30 alcohol esterified by C16 fatty acid) to cerotic acid (C26:0), the other major component of bee wax is 6:1. Ethyl esters are also present, the most abundant species being ethyl palmitate, ethyl tetracosanoate, and ethyl oleate (Jimenez J J et al., J Chromatogr A 2004, 1024, 147).

Lanolin or wool wax is secreted by sheep sebaceous glands and collected from crude wool by dilute alkali or detergent washing. Unwashed wool contains about 10-24% of a greasy material and a small proportion of salts of long-chain fatty acids. Lanolin contains fatty esters (14-24%), sterols and triterpene alcohol esters (45-65%), free alcohols (6-20%), sterols (cholesterol, lanosterol) and terpenes (4-5%). Hydroxylated fatty acids (mainly hydroxy palmitate) are found either free or esterified. Fatty acid chains have from 14 up to 35 carbon atoms, many of them having branched chains (iso or anteiso conformations). Its melting point is 35-42° C. The crude lanolin contains about 17% of primary alcohols and 9% of diols. Among monoalcohols, 9% have a normal chain, 38% belong to the iso series and 53% to the anteiso series. Two third of the diols belong to the iso series (Fawaz F et al., Ann Pharm Fr 1974, 32, 215). Among acids, 27% are a-hydroxylated, 5.2% are w-hydroxylated and 4.7% are poly-hydroxylated (Fawaz F et al., Ann Pharm Fr 1974, 32, 59).

Carnauba wax also called Brazil wax and palm wax, is a wax of the leaves of the palm Copernicia prunifera, a plant native to and grown only in the northeastern Brazilian states of Piauí, Ceará, and Rio Grande do Norte. Carnauba wax is obtained from the leaves of the carnauba palm by collecting and drying them, beating them to loosen the wax, followed by physical and/or chemical refining, e.g., filtration, centrifugation or bleaching, of the wax. Carnauba comprises primarily aliphatic wax esters (about 70-80 wt %), diesters of 4-hydroxycinnamic acid (about 10.0 wt %), ω-hydroxycarboxylic acids (about 7.0 wt %), and fatty acid alcohols (about 7 wt %). The compounds are predominantly derived from fatty acids and fatty alcohols in the C26-C30 range. A preferred preparation of carnauba wax is one that is refined to remove much of the free acids and free alcohols from the wax, thereby leaving a preparation comprising mostly of a mixture of wax esters.

### Other Compositional Components for Tear Film Stabilization

Nutritional compositions containing omega-3 fatty acids are known in the art. WO 2004/004599 A3 (Advanced Vision Research) discloses a method for treatment of a condition selected from the group consisting of dry eye, irritation of Meibomian glands, dysfunction of Meibomian glands, and dry mouth. The method comprises administration of a dietary supplement, which contains an omega-6 fatty acid containing oil and omega-3 rich oil, wherein the omega-3 rich oil has a high concentration of eicosapentaenoic acid (EPA) and a high concentration of docosahexaenoic acid (DHA). Ophthalmic compositions that are applied directly to the eye and contain omega-3 fatty acids are not well known primarily because it is difficult to develop a stable aqueous based vehicle formulation. The vehicle formulation described herein addresses this problem in the art.

Omega-3 as well as omega-6 fatty acids are known to metabolize in the body inter alia to prostaglandins PGE1 and PGE3, the latter of which have an anti-inflammatory profile. There has been growing evidence that the dry eye condition has an aetiology in inflammation. The ophthalmic composition comprises a concentration of total omega-3 fatty acids in a range from 0.4 to 5 percent by weight, or from 0.5 to 2 percent by weight, calculated as triglycerides. The omega-3 fatty acids of choice include eicosapentaenoic acid (EPA), docosahexaenoic acid (DHA) or a mixture of EPA and DHA. Other omega-3 fatty acids of interest can be selected from the group consisting of alpha-linolenic acid, stearidonic acid, docosapentaenoic acid and, of course, any one mixture of the omega-3 acids above.

In many of the preferred ophthalmic compositions described herein, cationic zinc, e.g., in the form of zinc sulphate, is included with an omega-3 fatty acid. The presence of zinc is believed to promote the anti-inflammatory effect of the compositions. The addition of zinc in the form of a zinc compound in ophthalmic compositions that include omega-3 fatty acids fatty acids promotes the conversion of these fatty acids into PGE1 and PGE3, leading to improved results in the treatment of the dry eye syndrome. For example, in one embodiment, the omega-3 fatty acids will have a high concentration of eicosapentaenoic acid (EPA) or a high concentration of docosahexaenoic acid (DHA) in combination with at least one zinc compound.

The ophthalmic composition can also comprise omega-6 fatty acids. The omega-6 fatty acids of choice include gamma-linolenic acid in a concentration in the range from 10 to 50 percent by weight, or from 15 to 25 percent by weight, of the total omega-6 fatty acids included in the composition. Other omega-6 fatty acids that may be present in the composition can be selected from the group consisting of linoleic acid, dihomo-gamma-linolenic acid, and combinations thereof. In many embodiments, the concentration of the omega-6 fatty acids is in a range from 0.05 to 1.5 percent by weight, or from 0.05 to 0.5 percent by weight, of the total composition.

As stated, the ophthalmic compositions described can provide ocular tissues with an extra supply of biochemical precursors for the biosynthesis of PGE₁ and PGE₃. One possible source of the biochemical precursors are the omega-3 fatty acids present as triglycerides in naturally derived oils that predominantly comprise the omega-3 fatty acids, eicosapentaenoic acid and docosahexaenoic acid. A natural source of omega-3 fatty acids include, but are not limited to, rapeseed oil, linseed oil, and fish oil. Moreover, an omega-6 fatty acid in the form of gamma-linolenic acid can be obtained from borage seed oil, evening primrose oil, and/or core oil of black currants.

In one embodiment, a weight ratio of omega-3 fatty acids calculated as triglycerides and gamma-linolenic acid is from 20:1 to 100:1, or from 50:1 to 60:1. It is believed that the omega-3 fatty acid eicosapentaenoic acid (EPA) competes for the enzyme delta-5-desaturase against the dihomo-gamma-linolenic acid derived from the class of omega-6 fatty acids. The greater concentration of EPA in the compositions compared can not only increase the synthesis of PGE₃, but also indirectly compete for the action of delta-5-desaturase resulting in a reduced synthesis of the undesired arachidonic acid, and consequently, a reduced synthesis of PGE₂. Moreover, the EPA can be converted to the desirable PEG₃ in a cyclooxygenase pathway. The addition of a zinc compound, and optionally the vitamins described herein, can have a positive and synergistic effect which is not yet completely understood. Overall, the compositions described can provide a positive influence on the relative ratios of inflammation mediators, i.e. the ratios of the desired anti-inflammatory and tear secretion improving PGE1 and PGE3 on the one hand and the undesired inflammation promoting PGE2 on the other hand.

In another embodiment, an ophthalmic composition, in addition to the mixture of wax esters, can include at least one vitamin selected from the group consisting of vitamin A, vitamin E. vitamin C, vitamin D, vitamin B6, vitamin B12, and any one mixture thereof. Lipophilic vitamin E or vitamin C may inter alia act as antioxidant, and in combination with the mixture of wax esters can contribute to the maintenance of the natural tear film in the eye and to the improvement of the supply of natural moisture to the eye.

The described formulation vehicle is used to provide a suspension of a mixture of wax esters in a vehicle formulation that comprises a lightly cross-linked carboxy-containing polymer and a concentration of ionic salt components to provide the suspension with a calculated ionic strength of less than 0.1. In addition, the suspension has the following rheological properties, G′>G″ and a suspension yield value of greater than 1 Pa. e.g., from 2 Pa to 10 Pa or from 3 Pa to 6 Pa. Also, upon addition of 30 mL of the suspension to a volume of 6 mL to 12 mL of simulated tear fluid, the resulting tear mixture transitions to a more liquid-state wherein, G″>G′ and the tear mixture has a yield value of less than 0.1 Pa, and more likely less than 0.05 Pa or less than 0.01 Pa. In fact, in many instances, the mixture will have no measurable yield value using the experimental methods described in Example I under the subheading, Experiments Rheology.

Viscoelasticity of a compositional fluid can in-part be described by what is referred to as a complex shear modulus defined by the following formula.
G*=G′+iG″ where i²=−1, and G′ is referred to as a storage modulus, and G″ is referred to as a loss modulus.
   G′ is often referred to as the elastic modulus or storage modulus and relates to the fluid's ability to store elastic energy. G″ is often referred to as the viscous modulus or loss modulus and relates to the fluid's viscosity or its ability to dissipate energy when a shear force is applied to the fluid. When G′>>G″, then the viscoelastic properties are dominated by the elastic properties and indicates that the composition is classified as a gel (semisolid). When G″>G′, then the viscoelastic properties are dominated by the viscous behavior and the composition is classified as a sol (liquid).

The relative magnitude of G′ and G″ can also be quantitated using an angle delta, δ, which is defined by tan δ=G″/G′. Under conditions where G′=G″, then tan δ=1 and δ=45 degrees. When G″ then tan δ<<1 and δ<<45 degrees. For vehicle formulations that exhibit the desired behavior of maintaining a physically stable suspension in the bottle, δ is less than 10° and tan δ less than 0.2. Many of the preferred vehicle formulations will exhibit a δ is less than 6°, e.g., from 2° to 5°, or a tan δ of less than 0.105, e.g., a tan δ from 0.035 to 0.0875, respectively. This requirement that tan δ be less than 0.2 ensures that G′ is at least 5 times greater than G″. Because the values of G′ and G″ may be affected by the oscillatory frequency used in the measurement, it is preferred that the values of δ and tan δ be measured at 1 rad/s.

The simulated tear fluid used to determine the rheological properties of the mixture is listed in Table 1 below. Hanks balanced salt solution (Gibco HBSS with calcium and magnesium. P/N 14025, Invitrogen Corp, Carlsbad, Calif.) was used as simulated tear fluid. Again, addition of the tear fluid is used to simulate the ocular environment of the suspension following instillation into the eye. If you calculate the ionic strength of the simulated tear fluid one obtains a value of 0.15. The ionic strength is essentially equivalent to the ionic strength of 0.9% saline, which is also calculated to be 0.15. This is expected because the simulated tear fluid is comprised of 0.8% saline, and the additional salts and buffer agents make a small contribution to the ionic strength to the solution.

In many embodiments, the suspension will have a viscosity in the container for instillation of eye drops of from 1000 cp to 2000 cp, and the calculated ionic strength is from 0.03 units to 0.1 units. Viscosity is measured with a Brookfield Engineering Laboratories LVDV-III Ultra C rheometer (a cone-and-plate rheometer) with CPE-52 spindle, at 25° C., and shear rate of 7±1 s⁻¹. Additional information on the viscosity measurements is described in the Example section.

TABLE 1

| Component | mg/L | mmol |
|---|---|---|
| Calcium chloride | 140 | 1.26 |
| Magnesium chloride | 100 | 0.493 |
| Magnesium sulfate | 100 | 0.407 |
| Potassium chloride | 400 | 5.33 |
| Potassium dihydrogen phosphate | 60 | 0.441 |
| Sodium hydrogen carbonate | 350 | 4.17 |
| Sodium chloride | 8000 | 138 |
| Disodium hydrogen phosphate | 48 | 0.338 |
| d-glucose | 1000 | 5.56 |

Controlling the ionic strength of the suspension with the formulation vehicle important to achieve the desired rheological properties. Accordingly, the suspension will have a calculated ionic strength from 0.03 units to 0.1 units, preferably from 0.05 units to 0.09 units.

The ionic strength of the formulation is calculated according to the standard equation that states:

μ=1/2ΣᵢCᵢzᵢ².

where the ionic strength, μ, is ½ the sum, over all charged species, i, of the product of the molar concentration, Cᵢ, and the square of the ion charge, zᵢ. The ionic strength is calculated using the equilibrium concentration of charged species at the pH of the formulation and not based solely on the formulation recipe. The equilibrium concentration of charged species for formulation components, at the pH of the formulation, can be estimated using the dissociation exponents, pK, for the ionizable species and the Henderson-Hasselbach equation. More preferably, the equilibrium concentration of charged species and ionic strength will be calculated simultaneously using an iterative approach similar to that outlined in Okamoto et al. [H. Okamoto, K. Mori, K. Ohtsuka, H. Ohuchi, and H. Ishii. Pharmaceutical Research, Vol. 14, No. 3, 1997] where a computer program is used to also include the effect of ionic strength on the pK. The contribution of the cross-linked carboxy-containing polymer to the ionic strength is included by treating the polymer as simply composed of acrylic acid with a monomer weight of 72 g/mol and a pKa of about 4.5. For example, at pH below the pKa, the polymer does not significantly contribute to the ionic strength, but at pH above the pKa, sodium acrylate (present when the polymer is neutralized by addition of sodium hydroxide) will contribute to the ionic strength.

The relatively fast transition from gel to liquid upon instillation of a described suspension into an eye is an important rheological characteristic of the ophthalmic formulation vehicle. We believe that this transition from gel to liquid may be triggered by the sudden change in ionic strength resulting from the dilution of the suspension with small amounts of tear fluid, particularly within the initial five minutes following instillation. The ionic strength of tear fluid is relatively quite high because of the high salt concentrations in tears. As the ophthalmic suspension mixes with the tear fluid the ionic strength of the resulting suspension-tear mixture, hereafter tear mixture, increases relative to the suspension and causes the suspension to thin. Also, because the concentration of the carboxy-containing polymer in the described vehicle formulation is typically less than the formulation vehicle of prior art drug suspensions the increase in the ionic strength upon dilution with tear fluid has greater affect and leads to greater tear thinning. The relatively large difference in the pre- and post-instillation ionic strength environments, and the relatively smaller amounts of carboxy-containing polymer in the vehicle formulations described herein, is believed to drive the thinning of the suspension in the eye.

As stated, the tear thinning characteristics of the described vehicle formulations is driven primarily by the differences in the ionic strength between the vehicle formulation, which has a low ionic strength relative to the high ionic strength of tear fluid, represented by the simulated tear fluid. As the vehicle formulation is administered in the form of an eye drop onto an eye, the percentage increase in the ionic strength of the tear mixture causes the vehicle formulation to thin. Accordingly, one can define a % ionic strength as a percent ratio of the ionic strength of the vehicle over the ionic strength of the simulated tear fluid, which is 0.154. Because the concentration of polymer also plays a role in the thinning characteristics of the vehicle formulations one can multiply the polymer concentration by the % ionic strength to give a tear thin value that can be used to predict with some confidence whether one will observe the desired tear thinning of a vehicle formulation.

% ionic strength=[formulation i.s./tear i.s.]×100

tear thin value=% ionic strength×[polymer, wt. %]

Accordingly, in one embodiment, an ophthalmic composition will comprise a mixture of wax esters suspended in a formulation vehicle. The vehicle will have from 0.3 wt. % to 0.5 wt. % of a cross-linked carboxy-containing polymer described below, and from 0.01 wt. % to 0.2 wt. % of sodium and/or potassium salts. The suspension can also include small amounts of bivalent calcium/magnesium chloride, which is known to have a somewhat greater affect on ionic strength. In many instances, the cross-linked carboxy containing polymer will be a poly(acrylic acid) type polymer, e.g., the polymers referred to in the art as polycarbophil or carbomer. The preferred weight ratio of carboxy polymer to salt is about 4:1 to 20:1, or from about 6:1 to about 12:1.

The lightly cross-linked carboxy-containing polymers for use in the present invention are lightly cross-linked polymers of acrylic acid or the like and are, in general, well-known in the art. See, for example, Robinson U.S. Pat. No. 4,615,697, and International Publication No. WO 89/06964. These polymers are also described by Davis et al in U.S. Pat. No. 5,192,535.

In a preferred embodiments of the formulation vehicle, suitable polymers are ones prepared from at least about 90% and preferably from about 95% by weight, based on the total weight of monomers present, of one or more carboxyl-containing monoethylenically unsaturated monomers. Acrylic acid is the preferred carboxyl-containing monoethylenically unsaturated monomer, but other unsaturated, polymerizable carboxyl-containing monomers, such as methacrylic acid, ethacrylic acid, β-methylacrylic acid (crotonic acid), cis-α-methylcrotonic acid (angelic acid), trans-α-methylcrotonic acid (tiglic acid), α-butylcrotonic acid, α-phenylacrylic acid, α-benzylacrylic acid, α-cyclohexylacrylic acid, β-phenylacrylic acid (cinnamic acid), coumaric acid (o-hydroxycinnamic acid), umbellic acid (p-hydroxycoumaric acid), and the like can be used in addition to or instead of acrylic acid.

The lightly cross-linked carboxy-containing polymers are prepared by using a small percentage, i.e., less than about 5%, such as from about 0.01% or from about 0.5% to about 5%, and preferably from about 0.2% to about 3%, based on the total weight of monomers present, of a polyfunctional cross-linking agent. Included among such cross-linking agents are non-polyalkenyl polyether difunctional cross-linking monomers such as divinyl glycol; 3,4-dihydroxy-hexa-1,5-diene; 2,5-dimethyl-1,5-hexadiene; divinylbenzene; N,N-diallylacrylamide; N,N-diallylmethacrylamide and the like.

The lightly cross-linked polymers can of course be made from a carboxyl-containing monomer or monomers as the sole monoethylenically unsaturated monomer present, together with a cross-linking agent or agents. They can also be polymers in which up to about 40%, and preferably from about 0% to about 20% by weight, of the carboxyl-containing monoethylenically unsaturated monomer or monomers has been replaced by one or more non-carboxyl-containing monoethylenically unsaturated monomers containing only physiologically (and, where appropriate, ophthalmologically) innocuous substituents, including acrylic and methacrylic acid esters such as methyl methacrylate, ethyl acrylate, butyl acrylate, 2-ethylhexylacrylate, vinyl acetate, 2-hydroxyethylmethacrylate, 3-hydroxypropylacrylate, and the like. Particularly preferred polymers are lightly cross-linked acrylic acid polymers wherein the cross-linking monomer is 3,4-dihydroxyhexa-1,5-diene or 2,5-dimethylhexa-1,5-diene.

An especially preferred lightly cross-linked carboxy-containing polymer for use herein is polycarbophil, particularly NOVEON AA1, a carboxyl-containing polymer prepared by suspension polymerizing acrylic acid and divinyl glycol. NOVEON AA1 (also called Carbopol 976) is commercially available from The B.F. Goodrich Company. A different preferred lightly cross-linked carboxy-containing polymer for use herein is Carbopol 974P which is prepared using a different polyfunctional cross-linking agent of the polyalkenyl polyether type. Still another class of lightly cross-linked carboxy-containing polymer are known in the art as carbomer, e.g., carbomer 940.

The lightly cross-linked polymers can be commercially available, or are generally preferably prepared by suspension or emulsion polymerizing the monomers, using conventional free radical polymerization catalysts. In general, such polymers will range in molecular weight estimated to be from about 250,000 to about 4,000,000, and preferably from about 500,000 to about 2,000,000.

In general, the present invention provides an ophthalmic formulation that is topically administrable into an eye of a subject as a drop. The described ophthalmic compositions takes advantage of the tear thin character of the vehicle formulation described herein. In one embodiment, the viscosity of the vehicle formulation does not increase upon contact with the tear fluid in the eye. The vehicle formulation is sufficiently viscous (>1000 cps at 7.5 s⁻¹ shear) to ensure the mixture of wax esters remains suspended in the vehicle and do not separate from the gel and coaleses over an extended period of time. The stabilized gel formulation does not require shaking of the dosage package to re-suspend the mixture of wax esters prior to drop administration.

The vehicle formulations described herein can also include various other ingredients, including but not limited to surfactants, tonicity agents, buffers, preservatives, co-solvents and viscosity-building agents.

Surfactants that can be used are surface-active agents that are acceptable for ophthalmic or otolaryngological uses. Useful surface active agents include but are not limited to polysorbate 80, tyloxapol, Tween® 80 (ICI America Inc., Wilmington. Del.), Pluronic® F-68 (from BASF, Ludwigshafen, Germany) and the poloxamer surfactants can also be used. These surfactants are nonionic alkaline oxide condensates of an organic compound which contains hydroxyl groups. The concentration in which the surface active agent may be used is only limited by neutralization of the bactericidal effects on the accompanying preservatives (if present), or by concentrations which may cause irritation.

Various tonicity agents may be employed to adjust the tonicity of the formulation. For example, sodium chloride, potassium chloride, magnesium chloride, calcium chloride, nonionic diols, preferably glycerol, dextrose and/or mannitol may be added to the formulation to approximate physiological tonicity. Such an amount of tonicity agent will vary, depending on the particular agent to be added. In general, however, the formulations will have a tonicity agent in an amount sufficient to cause the final formulation to have an ophthalmically acceptable osmolality (generally about 150-450 mOsm/kg). A nonionic tonicity agent is preferred. However, if an ionic compound is used to assist in adjusting the tonicity, such compound is used in an amount such that the total concentration of cations in a composition of the present invention is within the range herein disclosed.

An appropriate buffer system (e.g., sodium phosphate, sodium acetate, sodium citrate, sodium borate or boric acid) may be added to the formulations to prevent pH drift under storage conditions. The particular concentration will vary, depending on the agent employed.

Topical ophthalmic products are typically packaged in multidose form. Preservatives are thus required to prevent microbial contamination during use. Suitable preservatives include: biguanides, hydrogen peroxide, hydrogen peroxide producers, benzalkonium chloride, chlorobutanol, benzododecinium bromide, methyl paraben, propyl paraben, phenylethyl alcohol, edetate disodium, sorbic acid, polyquaternium-1, or other agents known to those skilled in the art. Such preservatives are typically employed at a level of from 0.001 to 1% (w/w). Unit dose forms will be sterile and generally will not contain preservatives.

Co-solvents and viscosity-building agents may be added to the formulations to improve the characteristics of the formulations. Such materials can include nonionic water-soluble polymer. Other compounds designed to lubricate, “wet,’ approximate the consistency of endogenous tears, aid in natural tear build-up, or otherwise provide temporary relief of dry eye symptoms and conditions upon ocular administration the eye are known in the art. Such compounds may enhance the viscosity of the formulation, and include, but are not limited to: monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as, polyvinyl alcohol, polyvinylpyrrolidone, povidone and carbomers, such as, carbomer 934P, carbomer 941, carbomer 940, carbomer 974P. Other compounds may also be added to the ophthalmic formulations of the present invention to increase the viscosity of the carrier. Examples of viscosity-enhancing agents include, but are not limited to: polysaccharides, such as hyaluronic acid and its salts, chondroitin sulfate and its salts, dextrans, various polymers of the cellulose family; vinyl polymers; and acrylic acid polymers.

Formulations formulated for the treatment of dry eye-type diseases and disorders may also comprise aqueous carriers designed to provide immediate, short-term relief of dry eye-type conditions. Such carriers can be formulated as a phospholipid carrier or an artificial tears carrier, or mixtures of both. As used herein, “phospholipid carrier” and “artificial tears carrier” refer to aqueous formulations which: (i) comprise one or more phospholipids (in the case of phospholipid carriers) or other compounds, which lubricate, “wet,” approximate the consistency of endogenous tears, aid in natural tear build-up, or otherwise provide temporary relief of dry eye symptoms and conditions upon ocular administration.

The ophthalmic compositions described herein are intended for administration to a human patient suffering from ophthalmic diseases such as dry eye or symptoms of dry eye. Preferably, the compositions will be administered topically. In general, the doses used for the above described purposes will vary, but will be in an effective amount to eliminate or improve dry eye conditions. Generally, 1-2 drops of such compositions will be administered from once to many times per day. The composition is intended to be provided as a package for the treatment of dry eye. In certain embodiments wherein the composition is preservative free, the package would contain a pharmaceutically acceptable container suitable for single use by a user. Preferably the outer packing would contain a multiplicity of single use containers, for example, enough single use containers to provide for a one-month supply of the composition. To minimize or prevent loss of water from the unit dosage forms the unit dosage forms can be foil wrapped into weekly package units.

The invention will now be further described by way of several examples that are intended to describe but not limit the scope of the invention as defined by the claims herein.

Representative eye drop compositions are provided by the Examples and Comparative Examples below. All experiments are conducted under bench-top laboratory conditions at room temperature (about 23° C.) unless a different temperature is expressly stated in the Example.

### EXAMPLE 1

A sterile, aqueous polyacrylic acid polymer solution is mixed with a sterile-filtrated solution of a mixture of wax esters extracted from jojoba, preserving agent, isotonicity agent, and chelating agent. After careful and thorough mixing of the starting materials, the addition of sterile-filtrated caustic soda solution initiates gel formation, and the gel is further subjected to agitation until it is homogenous. Alternatively, a mixture of the wax esters can be first dissolved or suspended in a small amount of mineral oil and added to the polyacrylic acid polymer solution. The resulting suspension is then conventionally decanted or drawn off under sterile conditions into sterile containers.

The gel suspension is well acceptable to the patient because upon instillation it does not have the undesired characteristics of known ointments and is not oily. Also, stability studies have shown so that the gel has a relatively long shelf life without any change in its physical properties. In particular, there is little, or no, separation of the mixture of wax esters from the gel upon storage (25-40° C.) for at least 2 months. This sterile gel suspension represents a significantly improved dosage form for ophthalmic applications.

TABLE 2
Jojoba Suspension Formulations

| | | Amount Range | |
|---|---|---|---|
| Ingredient | Ex. 1 | (per 100 g) | |
| Cross-linked carboxy polymer) | 0.375 g | 0.2-0.5 g; 0.3-0.4 g | |
| Purified water | 99.625 g | q.s. to 100 g of | |
| Propylene glycol | 0.44 g | 0.3-0.6 g; 0.4-0.5 g | |
| Glycerin | 0.88 g | 0.6-1 | g; |
| Jojoba wax esters | 0.5 g | 0.3-2 g; 0.1-0.5 g | |
| Mineral oil | 0.02 g | 0.005-0.1 | g |
| Edetate disodium dihydrate | 0.055 g | 0.03-0.07 | g |
| Tyloxapol | 0.05 g | 0.03-1 | g |
| Boric acid | 0.5 g | 0.3-0.6 | g |
| Sodium Chloride | 0.05 g | 0.0-0.07 | g |
| Benzalkonium chloride (“BAK”) | 0.006 g | 0.003-0.01 | g |

Mix the components of Example 1 for more than 15 minutes and adjust pH to 6.3-6.6 using 2N NaOH (for the foregoing formulation, about 1.6-1.7 g of 2N NaOH is adequate).

### Rheology Measurements

Test Materials: The rheological properties of lightly crosslinked carboxy polymer-based formulations are evaluated neat as well as following dilution with synthetic tear fluid. Hanks balanced salt solution (Gibco HBSS with calcium and magnesium, P/N 14025, Invitrogen Corp, Carlsbad, Calif.) is used as simulated tear fluid for the dilution of the formulation because it mimics the osmolality, pH, ionic strength, and buffer capacity of tear fluid and has representative levels of magnesium and calcium which could affect the viscosity of the crosslinked polyacrylic acid-based formulation upon dilution.

Rheology Instrument: A controlled stress rheometer (TA Instruments AR2000 with Firmware V7.20, New Castle, Del.) is used for the measurement of the rheological properties of the formulation. The measurement system includes a stainless steel vaned-rotor (P/N 545025.001) and aluminum concentric cylinder cup (P/N 545622.001) which requires approximately 30 mL of sample for each measurement. The temperature of the sample cup is controlled by a peltier jacket and was maintained at 25° C. for all the experiments. Data is collected using Rheology Advantage software V5.7.1.3 (TA Instruments, New Castle, Del.). The measurement gap is set to 4 mm and the gap closing method is set to ‘exponential’. After closing the gap, the sample is equilibrated for 10 minutes prior to running each experiment. Data is collected using Rheology Advantage software V5.7.13 (TA Instruments, New Castle, Del.).

### Oscillatory Frequency Sweep

A frequency sweep experiment is performed at a constant oscillatory strain of 1%, by scanning from 50 to 0.2 rad/s (log scale, 10 points/decade). Vehicle formulations comprised of crosslinked polyacrylic acid polymers generally have values of G′, δ, and tan δ that are relatively constant over this frequency range. For the characterization of the gel properties in the formulation or the tear mixture, the values of G′, δ, and tan δ at 1 rad/s are used.

### Steady State Flow

A steady-state flow experiment is performed by scanning the shear rate from 100 s⁻¹ to 0 s⁻¹ (log scale, 10 points/decade). Steady state equilibrium is defined as 3 consecutive measurements within the tolerance window of 2%. The sample period is 5 seconds and the maximum time/point is set to 10 minutes. The motor mode is set to ‘auto’. The viscosity of the formulation or the tear mixture is significantly higher at low shear rate and lower at high shear rate because of the shear-thinning behavior exhibited by crosslinked polyacrylic acid polymers. The yield value for the formulation or the tear mixture is determined from the plot of shear stress versus shear rate. The yield value may be determined graphically, but a preferred method is to fit the shear rate versus shear stress data to the Herschel-Bulkley equation and use the best-fit yield value. Fitting of the steady-state flow data, in the 10 s⁻¹ to 0 s⁻¹ range, to the Herschel-Bulkley equation is performed using the Rheology Advantage Data Analysis Program (v.5.7.0). In the case where the best-fit yield value was <0, the yield value is reported as zero.

### EXAMPLE 2

A vehicle formulation of the invention that includes a mixture of hyaluronic acid and jojoba is described in Table 3.

TABLE 3
Hyaluronate-Jojoba Suspension Formulations

| | | Amount Range | |
|---|---|---|---|
| Ingredient | Ex. 2 | (per 100 g) | |
| Cross-linked carboxy polymer) | 0.375 g | 0.2-0.5 g; 0.3-0.4 g | |
| Purified water | 99.625 g | q.s. to 100 g of | |
| Propylene glycol | 0.44 g | 0.3-0.6 g; 0.4-0.5 g | |
| Glycerin | 0.88 g | 0.6-1 | g; |
| Jojoba wax esters | 0.5 g | 0.3-2 g; 0.1-0.5 g | |
| Mineral oil | 0.02 g | 0.005-0.1 | g |
| Hyaluronic acid | 0.05 g | 0.005-0.1 | g |
| Edetate disodium dihydrate | 0.055 g | 0.03-0.07 | g |
| Tyloxapol | 0.05 g | 0.03-1 | g |
| Boric acid | 0.5 g | 0.3-0.6 | g |
| Sodium Chloride | 0.05 g | 0.0-0.07 | g |
| Benzalkonium chloride (“BAK”) | 0.006 g | 0.003-0.01 | g |

### EXAMPLE 3

A vehicle formulation of the invention that includes a mixture of phospholipid and jojoba is described in Table 4.

TABLE 4
Phopholipid-Jojoba Suspension Formulations

| | | Amount Range | |
|---|---|---|---|
| Ingredient | Ex. 3 | (per 100 g) | |
| Cross-linked carboxy polymer) | 0.375 g | 0.2-0.5 g; 0.3-0.4 g | |
| Purified water | 99.625 g | q.s. to 100 g of | |
| Propylene glycol | 0.44 g | 0.3-0.6 g; 0.4-0.5 g | |
| Glycerin | 0.88 g | 0.6-1 | g; |
| Jojoba wax esters | 0.5 g | 0.3-2 g; 0.1-0.5 g | |
| Mineral oil | 0.02 g | 0.005-0.1 | g |
| phospholipid | 0.05 g | 0.005-0.1 | g |
| Edetate disodium dihydrate | 0.055 g | 0.03-0.07 | g |
| Tyloxapol | 0.05 g | 0.03-1 | g |
| Boric acid | 0.5 g | 0.3-0.6 | g |
| Sodium Chloride | 0.05 g | 0.0-0.07 | g |
| Benzalkonium chloride (“BAK”) | 0.006 g | 0.003-0.01 | g |

### EXAMPLE 4

A vehicle formulation of the invention that includes a mixture of phospholipid and jojoba is described in Table 5.

TABLE 5
Omega-3/Jojoba Suspension Formulations

| | | Amount Range | |
|---|---|---|---|
| Ingredient | Ex. 3 | (per 100 g) | |
| Cross-linked carboxy polymer) | 0.375 g | 0.2-0.5 g; 0.3-0.4 g | |
| Purified water | 99.625 g | q.s. to 100 g of | |
| Propylene glycol | 0.44 g | 0.3-0.6 g; 0.4-0.5 g | |
| Glycerin | 0.88 g | 0.6-1 | g; |
| Jojoba wax esters | 0.5 g | 0.3-2 g; 0.1-0.5 g | |
| Omega-3 fatty acid | 0.05 g | 0.005-0.1 | g |
| Edetate disodium dihydrate | 0.055 g | 0.03-0.07 | g |
| Tyloxapol | 0.05 g | 0.03-1 | g |
| Boric acid | 0.5 g | 0.3-0.6 | g |
| Sodium Chloride | 0.05 g | 0.0-0.07 | g |
| Benzalkonium chloride (“BAK”) | 0.006 g | 0.003-0.01 | g |

To determine the tear thinning characteristics of Example 1, 30 mL of the composition is mixed with 10 mL of simulated tear fluid, As stated, it is believed that the thinning characteristics of the described vehicle formulations is driven primarily by the differences in the ionic strength between the vehicle formulation, which has a relatively low ionic strength, and the relatively high ionic strength of tear fluid, represented by the simulated tear fluid. As the vehicle formulation is administered in the form of an eye drop onto an eye, the percentage increase in the ionic strength of the vehicle-tear mixture causes the vehicle formulation to thin. The concentration of the lightly cross-linked carboxy polymer, the calculated ionic strength of the example (vehicle) formulation, and the percent ratio in ionic strengths of the vehicle formulation over the simulated tear fluid given by the equation below. The ionic strength of the simulated tear fluid is 0.154. Because the concentration of polymer also plays a role in the thinning characteristics of the vehicle formulations one can multiply the polymer concentration by the % ionic strength to give a tear thin value that can be used to predict with some confidence whether one will observe the desired tear thinning of a vehicle formulation.

% ionic strength=[formulation i.s./tear i.s.]×100

tear thin value=% ionic strength×[polymer, wt. %]

Some of the more preferred vehicle formulations of the invention will have a % ionic strength of 60% or less, e.g., from 20% to 60%, or a tear thin value of 30 or less, e.g., from 5 to 30, or from 10 to 25.

This invention has been described by reference to certain preferred embodiments; however, it should be understood that it may be embodied in other specific forms or variations thereof without departing from its special or essential characteristics. The embodiments described above are, therefore, considered to be illustrative in all respects and not restrictive, the scope of the invention being indicated by the appended claims rather than by the foregoing description.

What is claimed:

1. A suspension comprising a mixture of wax esters suspended in a formulation vehicle, the vehicle comprising a lightly cross-linked carboxy-containing polymer and a concentration of ionic salt components to provide the suspension with a calculated ionic strength of less than 0.1,
wherein the suspension has the following rheological properties, G′>G″ and a suspension yield value of greater than 1 Pa, and upon addition of 30 mL of the suspension to a volume of 6 mL to 12 mL of simulated tear fluid to provide a tear mixture of the suspension in a simulated ocular condition, the tear mixture has G″>G′ and a tear mixture yield value of less than 0.1 Pa.

2. A suspension comprising 0.1 wt. % or 2.0 wt. % a mixture of wax esters obtained from a natural source selected from the group consisting of jojoba wax, beeswax, lanolin and carnauba, suspended in a formulation vehicle, the vehicle comprising a lightly crosslinked carboxy-containing polymer and a concentration of ionic salt components to provide the suspension with a calculated ionic strength of from 0.03 to 0.08, wherein the suspension has the following rheological properties, G′>G″ and a suspension yield value of from 2 Pa to 8 Pa, and upon addition of 30 mL of the suspension to a volume of 10 mL of simulated tear fluid to provide a tear mixture of the suspension in a simulated ocular condition, the tear mixture has a tear mixture yield value from 0 Pa to 0.1 Pa and a tear thin value of from 5 to 30.

3. The suspension of claim 1 wherein the mixture of wax esters is obtained from a natural source selected from the group consisting of jojoba wax, beeswax, lanolin and carnauba.

4. The suspension of claims 1 or 2 wherein the mixture of wax esters is present at a concentration of from 0.1% or 2.0%.

5. The suspension of claims 1 to 4 further comprising an omega-3 fatty acid selected from the group consisting of alpha-linolenic acid, stearidonic acid, eicosapentaenoic acid, docosapentaenoic acid, docosahexaenoic acid, and any one mixture of the omega-3 acids.

6. The suspension of any one of claims 1 to 5 further comprising a mixture of omega-6 fatty acids selected from the group consisting of linoleic acid, gamma-linolenic acid, dihomo-gamma-linolenic acid, and combinations thereof, and the mixture of omega-6 fatty acids is present at a concentration of from 0.05 wt. % to 1.5 wt. %.

7. The suspension of any one of claims 1 to 6 wherein the carboxy-containing polymer is selected from the group consisting of polycarbophil and carbomer.

8. The suspension of any one of claims 1 to 7 wherein the suspension has a tan δ measured at 1 rad/s of from 0.035 to 0.105.

9. The suspension of any one of claims 1 to 8 wherein the suspension has a yield value of from 2 Pa to 10 Pa.

10. The suspension of any one of claims 1 to 9 wherein the tear mixture yield value is from 0 Pa to 0.05 Pa if 30 mL of the suspension is diluted with a volume of 6 mL of simulated tear fluid.

11. The suspension of any one of claims 1 to 9 wherein the tear mixture has no measurable yield value if 30 mL of the suspension is diluted with a volume of 10 mL of simulated tear fluid.

12. The suspension of claim 11 comprising a tear thin value of from 5 to 30 when the 30 mL of the suspension is diluted with a volume of 10 mL of the simulated tear fluid.

13. The suspension of claim 12 wherein the tear thin value is from 10 to 25.

14. The suspension of any one of claims 1 to 13 further comprising a non-polar lipid selected from squalene and a triacylglycerol, wherein the non-polar lipid is present at a concentration of from 0.01 wt. % or 0.5 wt. %.

15. The suspension of any one of claims 1 to 14 further comprising 0.01 wt. % or 0.5 wt. % dipalmitoylphosphatidylcholine.

16. The suspension of any one of claims 1 to 15 wherein the formulation vehicle comprises 0.2-0.5% of the carboxy-containing polymer, 0.3-0.6% propylene glycol, 0.6-1% glycerin, and water, wherein all percentages are in percent by weight of the suspension.

18. A unit dosage package for administration of an ophthalmic formulation in the form of an eye drop, the ophthalmic formulation comprising a mixture of wax esters suspended in a formulation vehicle, the formulation vehicle comprises a lightly crosslinked carboxy-containing polymer and a concentration of ionic salt components to provide the suspension with a calculated ionic strength of from 0.03 to 0.08, wherein the ophthalmic formulation has the following rheological properties, G′>G″, and a suspension yield value of from 2 Pa to 8 Pa, and upon addition of 30 mL of the suspension to a volume of 10 mL of simulated tear fluid to provide a tear mixture of the suspension in a simulated ocular condition, the tear mixture has a tear mixture yield value from 0 Pa to 0.1 Pa and a tear thin value of from 5 to 30.

19. The unit dosage form of claim 8 wherein the mixture of wax esters is obtained from a natural source selected from the group consisting of jojoba wax, beeswax, lanolin and carnauba.

20. The unit dosage form of claim 18 or 19 wherein the wax ester is present at a concentration of from 0.1% or 2.0%.
